(19) United States
(12) Patent Application Publication
(10) Pub. No.: US 2012/0220019 A1
(43) Pub. Date: Aug. 30, 2012

(54) AIR COLLECTOR WITH FUNCTIONALIZED ION EXCHANGE MEMBRANE FOR CAPTURING AMBIENT CO2

(76) Inventor: Klaus S. Lackner, Dobbs Ferry, NY (US)

(21) Appl. No.: 13/386,587

(22) PCT Filed: Jul. 23, 2010
(86) PCT No.: PCT/US10/43133

§ 371 Date: May 4, 2012

Related U.S. Application Data
(60) Provisional application No. 61/228,106, filed on Jul. 23, 2009.

(51) Int. Cl. B01D 53/14; C01F 11/18; C12N 1/12; C01D 7/00

(52) U.S. Cl. 435/257.1; 423/210; 423/220; 423/235; 423/243.01; 423/226; 423/421; 423/430

(57) ABSTRACT

Methods, systems, apparatuses and compositions for extracting selected gases from a gas stream are provided. In some embodiments the invention involve a process of bringing a gas stream in contact with a primary sorbent, releasing a selected gas from the primary sorbent to create a selected gas-enriched gas mixture, and bringing the selected gas-enriched gas mixture in contact with an aqueous solution. The aqueous solution absorbs the selected gas from the selected gas-enriched gas mixture. In some embodiments, the selected gas is carbon dioxide.

This application claims the benefit of U.S. Provisional Application No. 61/228,106 filed Jul. 23, 2009, which application is incorporated herein by reference.

There is compelling evidence to suggest that there is a strong correlation between the sharply increasing levels of atmospheric CO₂ with a commensurate increase in global surface temperatures. This effect is commonly known as Global Warming. Of the various sources of CO₂ emissions, there are a vast number of small, widely distributed emitters that are impractical to mitigate at the source. Additionally, large scale emitters such as hydrocarbon-fueled power plants are not fully protected from exhausting CO₂ into the atmosphere. Combined, these major sources, as well as others, have lead to the creation of a sharply increasing rate of atmospheric CO₂ concentration. Until all emitters are corrected at their source, other technologies are required to capture the increasing, albeit relatively low, background levels of atmospheric CO₂. Efforts are underway to augment existing emissions reducing technologies as well as the development of new and novel techniques for the direct capture of ambient CO₂. These efforts require methodologies to manage the resulting concentrated waste streams of CO₂ in such a manner as to prevent its reintroduction to the atmosphere.

The production of CO₂ occurs in a variety of industrial applications such as the generation of electricity power plants from coal and in the use of hydrocarbons that are typically the main components of fuels that are combusted in combustion devices, such as engines. Exhaust gas discharged from such combustion devices contains CO₂ gas, which at present is simply released to the atmosphere. However, as greenhouse gas concerns mount, CO₂ emissions from all sources will have to be curtailed. For mobile sources the best option is likely to be the collection of CO₂ directly from the air rather than from the mobile combustion device in a car or an airplane. One advantage of removing CO₂ from air is that it eliminates the need for storing CO₂ on the mobile device.

Extracting carbon dioxide (CO₂) from ambient air would make it possible to use carbon-based fuels and deal with the associated greenhouse gas emissions after the fact. Since CO₂ is neither poisonous nor harmful in parts per million quantities, but creates environmental problems simply by accumulating in the atmosphere, it is possible to remove CO₂ from air in order to compensate for equally sized emissions elsewhere and at different times.

The most daunting challenge for any technology to scrub significant amounts of low concentration CO₂ from the air involves processing vast amounts of air and concentrating the CO₂ with an energy consumption less than that that originally generated the CO₂. Relatively high pressure losses occur during the scrubbing process resulting in a large expense of energy necessary to compress the air. This additional energy used in compressing the air can have an unfavorable effect with regard to the overall carbon dioxide balance of the process, as the energy required for increasing the air pressure may produce its own CO₂ that may exceed the amount captured negating the value of the process.

Various methods and apparatus have been developed for removing CO₂ from air. However, these methods result in the inefficient capture of CO₂ from air because these prior art methods heat or cool the air, or change the pressure of the air by substantial amounts. As a result, the net reduction in CO₂ is negligible as the capture process may introduce CO₂ into the atmosphere as a byproduct of the generation of electricity used to power the process. The present invention resolves these issues.

In some embodiments, the invention provides a method for extracting a selected from a gas stream by bringing the gas stream in contact with a primary sorbent and releasing the selected gas from the primary sorbent to create a selected gas enriched mixture. In some embodiments, the selected gas is selected from the group consisting of CO₂, NOₓ, and SO₂ In some embodiments, the selected gas is CO₂.

In some embodiments, the invention provides a method for extracting carbon dioxide from a gas stream by bringing the gas stream in contact with a primary sorbent and releasing the carbon dioxide from the primary sorbent to create a carbon dioxide-enriched gas mixture. The enriched gas mixture is then brought in contact with an aqueous solution where the aqueous solution absorbs carbon dioxide from the gas mixture.

In some embodiments, there is a gaseous gap between the primary sorbent and the aqueous solution. In some embodiments, the aqueous solution does not come into direct contact with the primary sorbent material.

In some embodiments, the carbon dioxide-enriched gas mixture is brought in contact with the aqueous solution by bubbling the carbon dioxide-enriched gas mixture through the aqueous solution. In some embodiments, the aqueous solution is flowed over surfaces that allow the aqueous solution to absorb carbon dioxide from the carbon dioxide-enriched gas mixture.

In some embodiments, the aqueous solution is water and is in contact with minerals from which alkali ions can be extracted. In some embodiments, the water is undersaturated in carbonate ions. In some embodiments, the water is continuously acidified with CO₂ in order to accelerate the dissolution of alkali ions.

In some embodiments, the aqueous solution may be an alkaline brine formed by seawater that is held in contact with a rock material containing carbonate or other materials from which alkali ions can be leached during its exposure to the CO₂. In some embodiments, the leached ion is a calcium ion. In some embodiments, at least part of the carbon dioxide is sequestered in the alkaline brine by forming carbonate ions, bicarbonate ions or a combination thereof, thereby neutralizing the aqueous solution, and further comprising returning the aqueous solution to its origin. In some embodiments, the alkaline brine that sequesters carbon dioxide is discharged into a body of ocean water where it mixes with the ocean water and adds a stable bicarbonate salt that sequesters carbon dioxide.

In some embodiments, the primary sorbent is an ion exchange resin. In some embodiments, the CO₂ may be transferred into a first aqueous wash which is separated from the aqueous solution by a gas diffusion membrane which allows the transfer of CO₂ from one side of the membrane to the other. In some embodiments, the aqueous solution is contained in or flows through a sponge or foam.

In some embodiments, the invention provides a composition comprising a CO₂ sequestering product, where the CO₂ sequestering product comprises carbon from ambient CO₂ from a gas mixture released from a primary sorbent. In some embodiments, the CO₂ sequestering product is a carbonate compound composition, a hydroxide composition, a bicarbonate composition, or a mixture thereof. In some embodiments, the carbonate compound composition comprises a precipitate from an alkaline-earth metal-containing water. In some embodiments, the δ¹³C is about 3‰ to about −35‰. In some embodiments, the ¹⁴C isotopic fraction is about 0.05 parts per trillion to about 2 parts per trillion. In some embodiments, the CO₂ sequestering product ranges from about 1% to about 5% w/w. In some embodiments the CO₂ sequestering product ranges from about 5 to 75% w/w. In some embodiments, the percentage of CO₂ in said gas mixture is about 1% to about 10%. In some embodiments, the percentage of CO₂ in said gas mixture is about 90% to about 100%. In some embodiments, the composition is used to store CO₂, feed algae, or dissolve alkaline metals. In some embodiments, the composition is used to store CO₂ in the ocean.

All publications and patent applications mentioned in this specification are herein incorporated by reference to the same extent as if each individual publication or patent application was specifically and individually indicated to be incorporated by reference.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

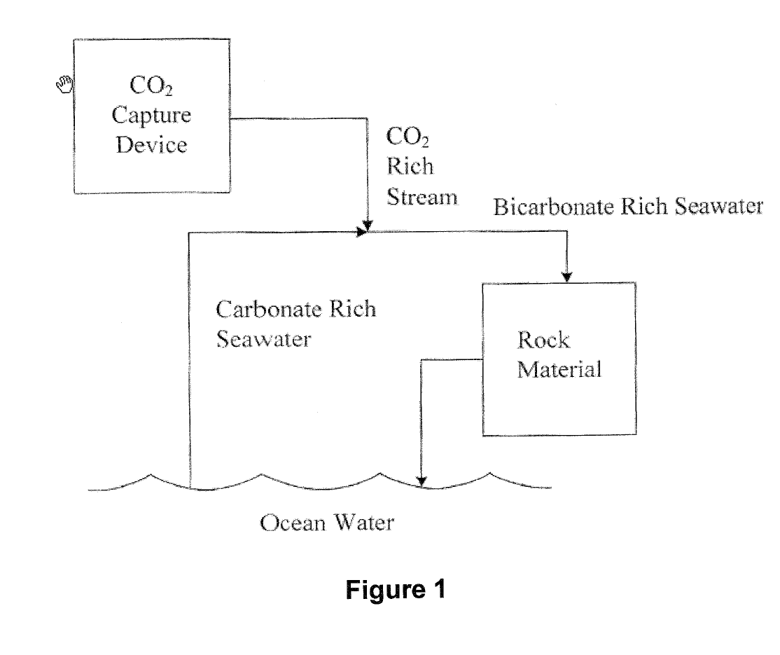

FIG. 1 is a schematic of an exemplary method for capture and sequestration of carbon dioxide.

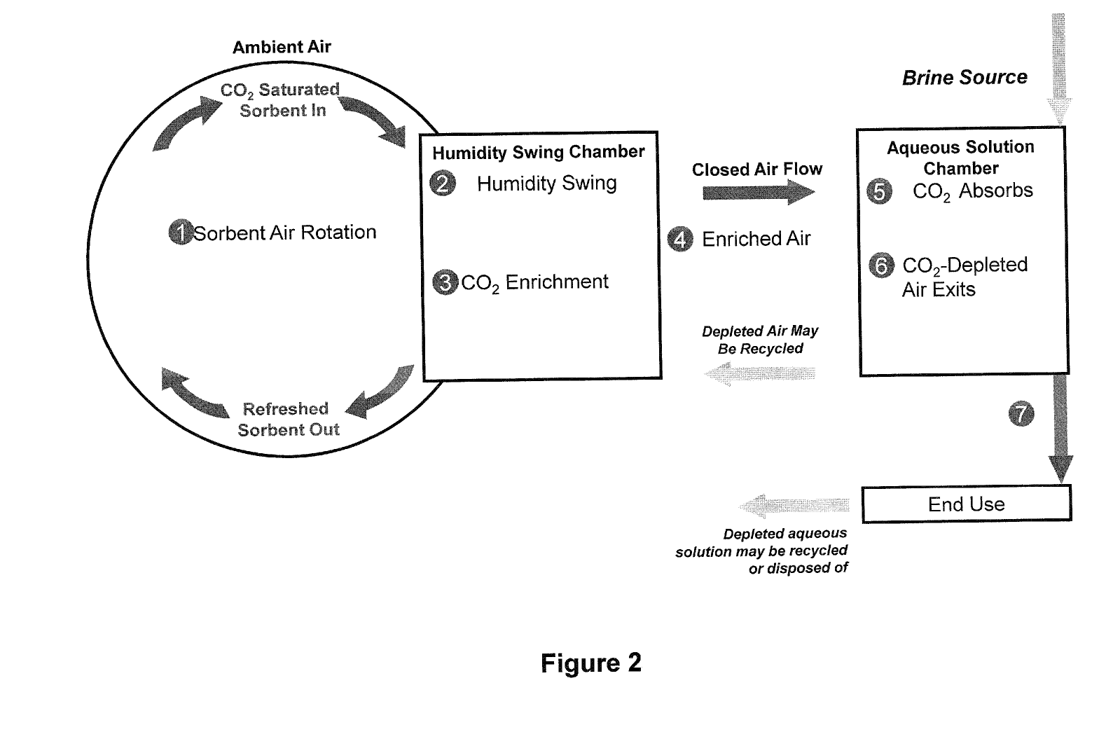

FIG. 2 is a schematic of an exemplary embodiment of the capture and sequestration of carbon dioxide.

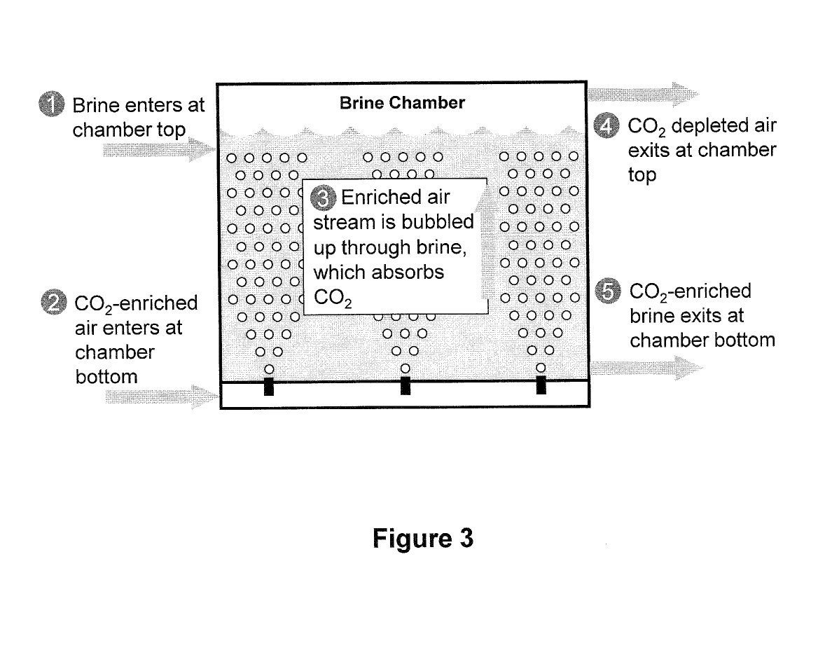

FIG. 3 is a schematic of an exemplary brine chamber in which enriched air is bubbled through brine.

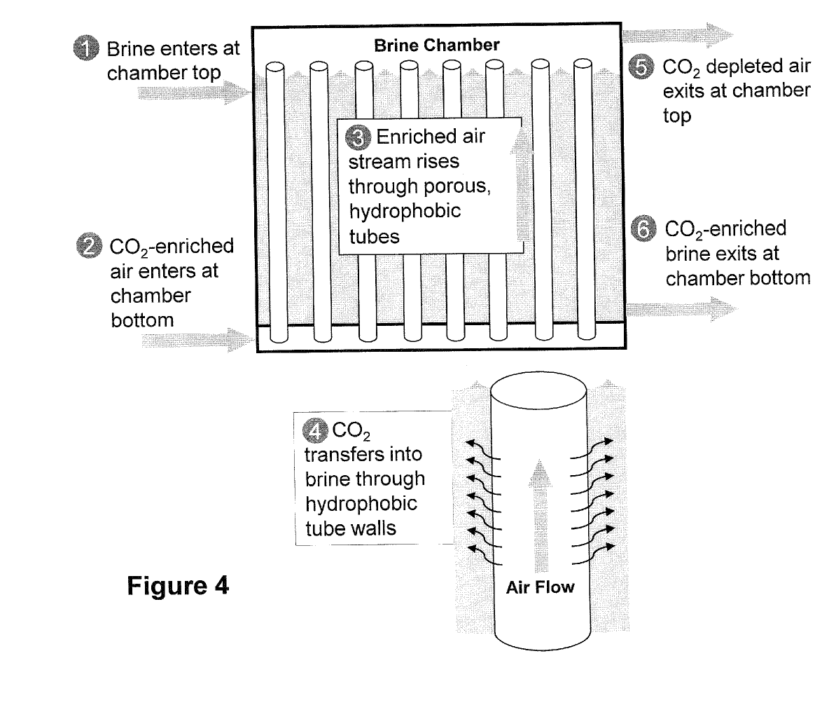

FIG. 4 is a schematic of an exemplary brine chamber where CO₂ is transferred to brine through hydrophobic tubes.

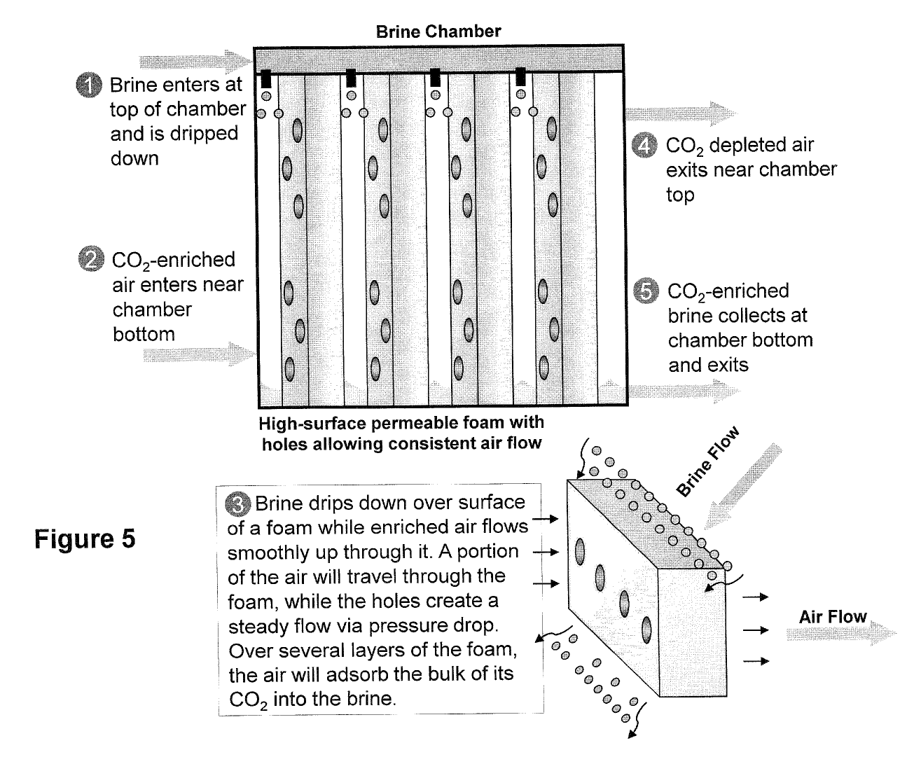

FIG. 5 is a schematic of an exemplary brine chamber where CO₂ is transferred to brine through foam across which brine is dripped.

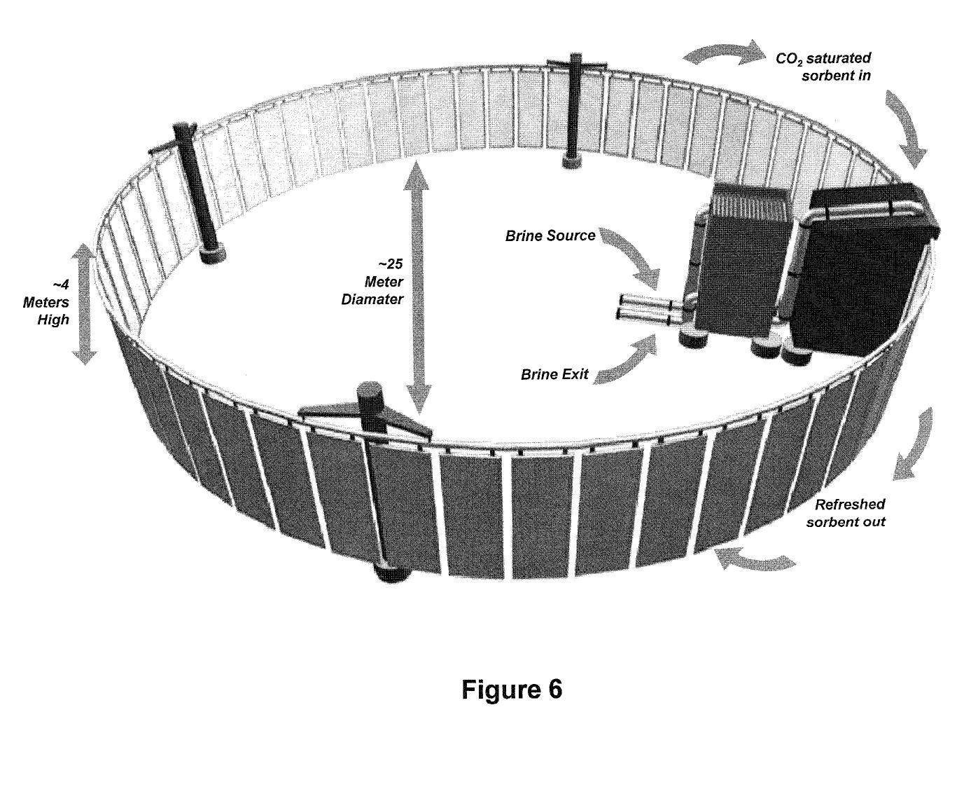

FIG. 6 is a schematic view of an exemplary device.

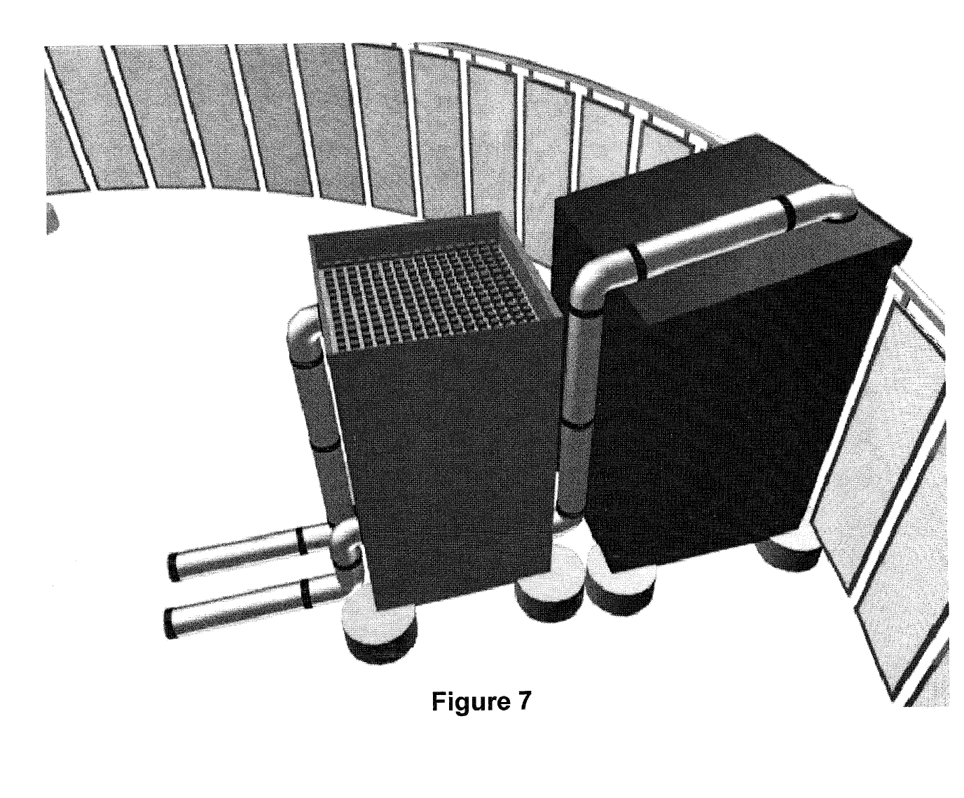

FIG. 7 is another schematic view of the exemplary device.

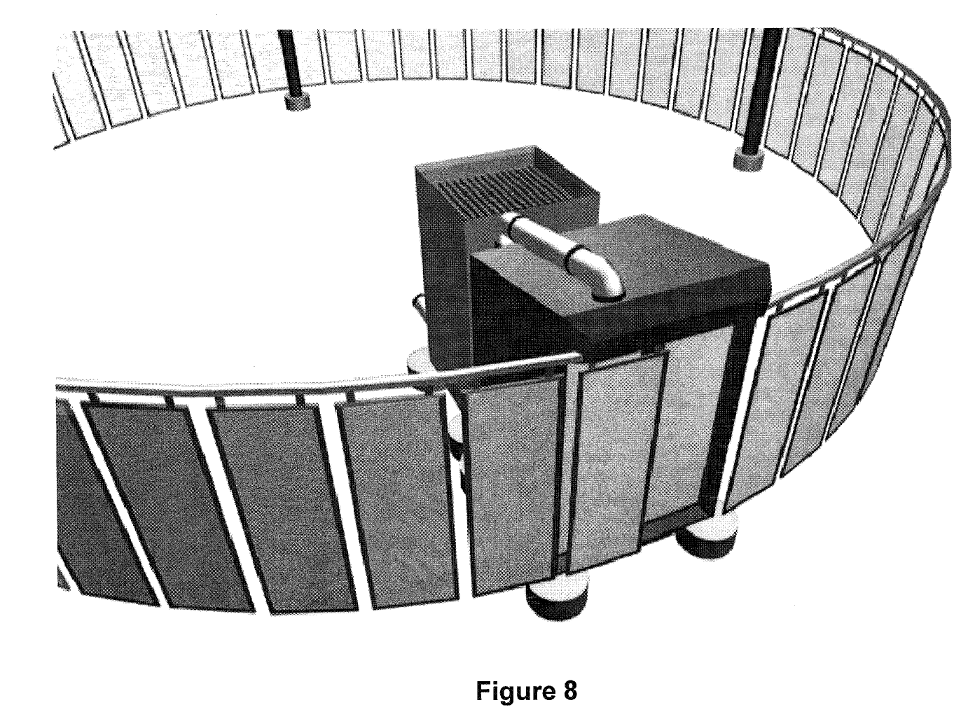

FIG. 8 is another schematic view of the exemplary device.

Reference will now be made in detail to particularly preferred embodiments of the invention. Examples of the preferred embodiments are illustrated in the following Examples section.

The present disclosure relates to removal of selected gases from a gas stream, e.g ambient air. In some embodiments, the disclosure have particular utility for the extraction of carbon dioxide (CO₂) from ambient air and will be described in connection with such utilities. In some embodiments, the invention generates a gas mixture that contains CO₂ and the CO₂ is then absorbed into an aqueous solution. Other utilities besides the extraction of CO₂ are contemplated, including the extraction of other gases including NOₓ and SO₂.

In some embodiments, the invention provides for methods, systems, apparatus and compositions for extracting selected gases (e.g. CO₂) from a gas stream. In some embodiments, the methods for extracting selected gases (e.g. CO₂) from a gas stream comprise bringing the gas stream in contact with a primary sorbent which temporarily binds the selected gas, releasing the selected gas from the primary sorbent to create a selected gas-enriched gas mixture, and bringing the selected gas-enriched gas mixture in contact with an aqueous solution, wherein the aqueous solution preferentially absorbs the selected gas from the selected gas-enriched gas mixture.

In some embodiments, the invention provides an air capture filter and method of forming said air capture filter using the materials described herein or other suitable materials currently available.

In some embodiments, the invention provides an advantageous method for capture and sequestration of carbon dioxide materials.

In some embodiments, the methods for extracting CO₂ from a gas stream comprise bringing the gas stream in contact with a primary sorbent, releasing CO₂ from the primary sorbent to create a CO₂-enriched gas mixture, and bringing the CO₂-enriched gas mixture in contact with an aqueous solution, wherein the aqueous solution absorbs CO₂ from the CO₂-enriched gas mixture. In some embodiments, the primary sorbent is located in a resin. A practical challenge in transferring CO₂ from a resin containing the primary sorbent to an aqueous solution (that is adapted for a subsequent use of CO₂) is the ability to have the CO₂ released from the primary sorbent without the aqueous solution touching the primary sorbent. That is that the aqueous solution may contain ions or impurities that should not get in contact with the resin containing the primary sorbent. For example, in some embodiments a seawater brine allows for the injection of CO₂ into seawater. But the chloride ion may not come in touch with an ionic exchange resin. Similarly, it is possible to feed CO₂ to algae by adding it to the brine, but this brine cannot be brought into direct contact with the sorbent. The set of inventions discussed here are concerned with this step and it considers number of applications that would be well served by such a system. Thus in some embodiments, the invention provides methods, apparatus and systems for extracting CO₂ from a gas stream by generating a gas mixture that contains CO₂ from a primary sorbent and absorbing the CO₂ from the gas mixture into an aqueous solution, where there is a gaseous gap between the primary sorbent and the aqueous solution. This gaseous gap protects the primary sorbent (e.g. an anionic exchange resin) for example, from ions and other impurities that may be present in the aqueous solution. Thus, in some embodiments the aqueous solution does not come in direct contact with the primary sorbent.

In some embodiments, the invention provides for compositions that include a selected gas sequestering product (e.g. CO₂ sequestering product), wherein the selected gas sequestering product comprises a chemical element from gas that was released from a gas mixture enriched for that selected gas (e.g. CO₂). In some embodiments, the invention provides compositions that include a selected gas sequestering product, wherein the selected gas sequestering product comprises a chemical element from gas that was released from a gas mixture enriched with certain relative element isotope composition. In some embodiments, the invention provides for compositions that include a selected gas sequestering product (e.g. CO₂ sequestering product), wherein the selected gas sequestering product comprises a chemical element from a gas that was released from gas mixture enriched for that selected gas (e.g. CO₂) and wherein the gas mixture is enriched with certain relative element isotope composition. In some embodiments, the gas mixture is a low pressure gas mixture. By “selected gas sequestering product” is meant that the product contains at least one chemical element (e.g. carbon) derived from a selected gas (e.g. CO₂).

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims. For the purpose of clarity and convenience only, the invention will be described mostly in terms of CO₂ sequestration; however, as described above sequestration of other gases are contemplated in the present invention.

Where a range of values is provided, it is understood that each intervening value to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a,” “an,” and “the” include plural references unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Primary Sorbent Material

The present invention provides for methods, systems, apparatus and compositions for the extraction or removal of selected gases from an air stream, e.g. ambient air using a sorbent material.

The present disclosure may be realized in connection with a broad range of sorbent materials for capturing any number of contaminants in a fluid stream, including for example hydrogen sulfide (H₂S) and bacteria. Other sorbents include methanol, sodium carbonate, weak liquid amine or hydrophobic activated carbon.

In some embodiments, the present invention extracts carbon dioxide from ambient air using a conventional CO₂ extraction method.

In some embodiments, the present invention extracts carbon dioxide from a gas stream using air scrubber units as described in PCT Application Nos. PCT/US05/29979. The air scrubber units remove CO₂ from an airflow that is maintained by a low pressure gradient. The air scrubber units can consist of a wind collector having lamella, which are two or more sheets or plates covered in liquid sorbent (which may or may not be downward flowing) bounding a thin air space, and a liquid sump. They sheets or plates could also be made from a solid sorbent. The sheets forming the lamella preferably are separated by spacers laced between the sheets on thru-rods supported by a rigid frame although the lamella may be supported in spaced relation by other means.

In general, the sorbent material flows down the lamella sheets, while the airflow passes between the thin airspace between the sheets. The contact between the air and the sorbent material causes a chemical reaction that removes CO₂. However, the air scrubber units could also capture other gases present in the air.

In some embodiments, the present invention extracts carbon dioxide from a gas stream using ion exchange materials to capture or absorb CO₂ as described in PCT/US06/029238. The ion exchange material can be a solid anionic exchange membrane as the primary CO₂ capture matrix. The ion exchange material may comprise a solid matrix formed of or coated with an ion exchange material. Alternatively, the material may comprise a cellulose based matrix coated with an ion exchange material.

In some embodiments, the invention employs a wetted foam air exchanger that uses a sodium or potassium carbonate solution, or other weak carbon dioxide sorbent, to absorb carbon dioxide from the air to form a sodium or potassium bicarbonate. The resulting sodium or potassium bicarbonate is then treated to refresh the carbonate sorbent which may be recovered and disposed of while the sorbent is recycled.

In some embodiments of the invention, carbon dioxide is removed from the air using an ion exchange material which is regenerated using a liquid amine solution which is then recovered by passing the amine solution into an electrodialysis cell.

In some embodiments, the present invention extracts carbon dioxide from a gas stream using anion exchange materials formed in a matrix exposed to a flow of the air, humidity swing or electrodialysis as described in PCT/US07/80229. In this process concentration enhancements of factors from 1 to 100 can be achieved. In some embodiments, concentration enhancements of factors of 100, 200, 300, 500, 600, 700, 800, or 900 can be achieved.

In one approach to CO₂ capture, the resin medium is regenerated by contact with the warm highly humid air. It has been shown that the humidity stimulates the release of CO₂ stored on the storage medium and that CO₂ concentrations between 3% and 10% can be reached by this method, and in the case of an evacuated system, a CO₂ concentration in the low pressure gas of close to 100% can be reached. In this approach the CO₂ is returned to gaseous phase and no liquid media are brought in contact with the collector material.

In some embodiments, the CO₂ extractor preferably comprises a humidity sensitive ion exchange resin in which the ion exchange resin extracts CO₂ when dry, and gives the CO₂ up when exposed to higher humidity. A humidity swing may be best suited for use in arid climates; however, it can be used in all kind of climates. Ion exchange resins are commercially available and are used, for example, for water softening and purification. Applicants have found that certain commercially available ion exchange resins which are humidity sensitive ion exchange resins and comprise strong base resins, advantageously may be used to extract CO₂ from the air in accordance with the present invention. Common commercially available ion exchange resins are made up of a polystyrene or cellulose based backbone which is aminated into the anionic form usually via chloromethalation. Once the amine group is covalently attached, it is now able to act as an ion exchange site using its ionic attributes. However, there are other ion-exchange materials and these could also be used for collection of CO₂ from the atmosphere. Examples of commercially available ion exchange resin that can be used in the methods, apparatuses and systems described herein include, but are not limited to, Anion I-200 from Snowpure, LLC, Type I and II functionality ion exchange from Dow, DuPont and Rohm and Hass. With such materials, the lower the humidity, the higher the equilibrium carbon dioxide loading on the resin.

Thus, a resin which at high humidity level appears to be loaded with CO₂ and is in equilibrium with a particular partial pressure of CO₂ will exhale CO₂ if the humidity is increased and absorb additional CO₂ if the humidity is decreased. The effect is large, and can easily change the equilibrium partial pressure by several hundred to several thousand ppm. This is useful in applications that involve photosynthetic organisms grown in commercial greenhouses or in algae ponds or algae reactors. If the gas volume is sufficiently constraint it is even possible to drive the CO₂ concentration in the gas up into ranges of 50,000 to 100,000 ppm. In some embodiments, CO₂ is released by wetting the resin material with liquid water. The additional take up or loss of carbon dioxide on the resin is also substantial if compared to its total uptake capacity.

The resins disclosed in our previous U.S. Provisional Patent Appln. 60/985,586 and PCT International Patent Appln. Serial No. PCT/US08/60672, assigned to a common assignee, make it possible to capture CO₂ from the air and drive it off the sorbent with no more than excess water vapor or liquid water.

The invention also encompasses other extraction processes, described in the prior art or disclosed herein, that releases at least a portion of the extracted CO₂ to a secondary process employing CO₂. The CO₂ also may be extracted from an exhaust at the exhaust stack.

The present invention provides a gaseous intermediary which is then immediately recaptured into an aqueous solution (e.g. brine). In some embodiments, the present invention provides a gaseous gap. The gaseous gap protects the sorbent material from impurities the aqueous solution might supply. The gaseous gap between the two materials can be quite large, or it can be managed quite tightly. The size of the gap will depend by the flow patters in a specific system. For instance, in some embodiments the gap will need to be large enough to avoid the accidental co-mingling of the sorbent, or the water that release the CO₂ from the sorbent, and the potentially “dirty” aqueous solution (e.g. brine).

In some embodiments, a gap of centimeters to tens of centimeters would be useful. In some embodiments, the gap is about 1, 2, 3, 4, 5, 10, 15, 20, 30, 40 or 50 centimeters. For example, one could alternate lamella sheets that involve different fluids, one being the aqueous solution (e.g. brine). In another example, a wider separation is obtained in a system where air is passing through foam blocks that are releasing CO₂ (e.g. because they are wetted by clean water), and blocks where the CO₂ is reabsorbed onto the aqueous solution (e.g. brine). The gaseous gap is maintained by a forward flow of the gas that will transfer the gas from one block of foam to the next.

In some embodiments, gap sizes are about 1, 2, 3, 4, 5, 10, 15, 20, 30, 40, or 50 millimeters. In some embodiments, gap sizes are about 1, 2, 3, 4, 5, 10, 15, 20, 30, 40, or 50 micrometers. For example, the gaps are small when the resin has a hydrophobic resin layer as discussed below, as the gaps are embedded into these very thin layers which may only be about 1, 2, 3, 4, 5, 10, 15, 20, 30, 40, or 50 micrometers thick.

In addition, the present invention provides utility and destination for extracted CO₂. As discussed below, some applications relate to immediate use of low grade CO₂, others refer to the long term or mediate term storage of CO₂ in an aqueous solution (e.g. brine). The extracted CO₂ can be delivered to controlled environments, e.g., greenhouses or in algae cultures. CO₂ may also be disposed of by using the extracted CO₂ to dissolve lime stone, thereby producing a calcium bicarbonate enriched brine that can be disposed of in the ocean.

### Aqueous Solution

In some embodiments, the present invention provides an aqueous solution that binds a selected gas (e.g. CO₂). In some embodiments, the aqueous solution recaptures a gaseous intermediary that has been released from a primary sorbent. In some embodiments, the aqueous solution recaptures CO₂ that has been released from a primary sorbent. In some embodiments, the aqueous solution is of sufficiently high pH to absorb CO₂ directly from a gaseous state released from a primary sorbent material. Without intending to be limited to any theory, the aqueous solution binds CO₂ more strongly than the sorbent material (e.g. humidity swing in its wet state). As a result CO₂ can be transferred from one material (e.g., the CO₂ absorbing resin filter in its wet state) to the other (e.g. the brine that readily absorbs CO₂).

In some embodiments, the aqueous solution is specific for the sorbent material (e.g. humidity swing) utilized. In some embodiments, the aqueous solution is specific for an intended use, e.g., store CO₂ in seawater or feed algae. In some embodiments, the aqueous solution is a synthetic composition that selectively absorbs CO₂ or any other selected gas. In some embodiments, the aqueous solution contains carbonates and/or bicarbonates. In some embodiment, the aqueous material is brine containing other anions and various cations. In some embodiments, the aqueous material is a bicarbonate brine. Examples of brines include but are not limited to sodium hydroxide, calcium hydroxide brine, and carbonate brine. The brine can be concentrated or diluted. Carbonate brines can be as diluted as 0.01 molar, or as concentrated as 5 to 10 molars. In some embodiments, the brine has a concentration of about 0.01, 0.03, 0.05, 0.10, 0.15, 0.30, 0.40, 0.5, 1, 2, 3, 4, 5, 6, 8, 10 or 15 molars.

In some embodiments, the aqueous solution is an aqueous solution of divalent cations. In some embodiments, the aqueous solution is an aqueous solution of monovalent cations. Divalent and monovalent cations may come from any of a number of different cation sources. Such sources include industrial wastes, seawater, brines, hard waters, rocks and minerals (e.g., lime, periclase, material comprising metal silicates such as serpentine and olivine), and any other suitable source. For example, in some embodiments strongly alkaline solutions of monovalent ions can be used, e.g., the bauxite sludges that result from the Bayer process in making alumina. These brines would be rich in sodium hydroxide.

In some embodiments, industrial waste streams from various industrial processes provide for convenient sources of cations, which are useful, for example, in the disposal of carbonates or bicarbonate brines. Such waste streams include, but are not limited to, mining wastes; fossil fuel burning ash (e.g., combustion ash such as fly ash, bottom ash, boiler slag); slag (e.g. iron slag, phosphorous slag); cement kiln waste; oil refinery/petrochemical refinery waste (e.g. oil field and methane seam brines); coal seam wastes (e.g. gas production brines and coal seam brine); paper processing waste; water softening waste brine (e.g., ion exchange effluent); silicon processing wastes; agricultural waste; metal finishing waste; high pH textile waste; and caustic sludge. Ash from the burning of fossil fuels, cement kiln dust, and slag, collectively waste sources of metal oxides, further described in U.S. patent application Ser. No. 12/486,692, filed 17 Jun. 2009, the disclosure of which is incorporated herein in its entirety. Any of the divalent and monovalent cations sources described herein may be mixed and matched for the purpose of practicing the invention. For example, material comprising metal silicates (e.g. serpentine, olivine), which are further described in U.S. patent application Ser. No. 12/501,217, filed 10 Jul. 2009, which application is herein incorporated by reference, may be combined with any of the sources of cations described herein for the purpose of practicing the invention. One advantage of divalent cation sources is that the resulting carbonates tend to have low solubility in water and thus are likely to precipitate out.

In some embodiments, a source of cations for preparation of a composition of the invention is water (e.g., an aqueous solution comprising cations such as seawater or surface brine), which may vary depending upon the particular location at which the invention is practiced. Suitable aqueous solutions of cations that may be used include solutions comprising one or more cations, e.g., alkaline earth metal cations such as Ca²⁺and Mg²′. In some embodiments, the aqueous solution of cations comprises cations in amounts ranging from 50 to 50,000 ppm, 50 to 40,000 ppm, 50 to 20,000 ppm, 100 to 10,000 ppm, 200 to 5000 ppm, or 400 to 1000 ppm. In some embodiments, the aqueous solution of cations comprises a mixture of two or more cations. In some embodiments, the aqueous source of cations comprises alkaline earth metal cations. In some embodiments, the alkaline earth metal cations include calcium, magnesium, or a mixture thereof. In some embodiments, the aqueous solution of cations comprises calcium in amounts ranging from 50 to 50,000 ppm, 50 to 40,000 ppm, 50 to 20,000 ppm, 100 to 10,000 ppm, 200 to 5000 ppm, or 400 to 1000 ppm. In some embodiments, the aqueous solution of cations comprises magnesium in amounts ranging from 50 to 40,000 ppm, 50 to 20,000 ppm, 100 to 10,000 ppm, 200 to 10,000 ppm, 500 to 5000 ppm, or 500 to 2500 ppm. In some embodiments, where Ca²⁺ and Mg⁺² are both present, the ratio of Ca²⁺ to Mg.²⁺ (i.e., Ca²⁺;Mg²⁺) in the aqueous solution of cations is between 1:1 and 1:2.5; 1:2.5 and 1:5; 1:5 and 1:10; 1:10 and 1:25; 1:25 and 1:50; 1:50 and 1:100; 1:100 and 1:150; 1:150 and 1:200; 1:200 and 1:250; 1:250 and 1:500; 1:500 and 1:1000, or a range thereof. For example, in some embodiments, the ratio of Ca²⁺ to Mg²⁺ in the aqueous solution of cations is between 1:1 and 1:10; 1:5 and 1:25; 1:10 and 1:50; 1:25 and 1:100; 1:50 and 1:500; or 1:100 and 1:1000. In some embodiments, the ratio of Mg²⁺ to Ca²⁺ (i.e., Mg.²⁺:Ca²⁺) in the aqueous solution of cations is between 1:1 and 1:2.5; 1:2.5 and 1:5; 1:5 and 1:10; 1:10 and 1:25; 1:25 and 1:50; 1:50 and 1:100; 1:100 and 1:150; 1:150 and 1:200; 1:200 and 1:250; 1:250 and 1:500; 1:500 and 1:1000, or a range thereof. For example, in some embodiments, the ratio of Mg²⁺ to Ca²⁺ in the aqueous solution of cations is between 1:1 and 1:10; 1:5 and 1:25; 1:10 and 1:50; 1:25 and 1:100; 1:50 and 1:500; or 1:100 and 1:1000, These ratios also apply to mixture of other cations.

The aqueous solution of cations may comprise cations derived from freshwater, brackish water, seawater, or brine (e.g., naturally occurring brines or anthropogenic brines such as geothermal plant wastewaters, desalination plant waste waters), as well as other salines having a salinity that is greater than that of freshwater, any of which may be naturally occurring or anthropogenic. Brackish water is water that is saltier than freshwater, but not as salty as seawater. Brackish water has a salinity ranging from about 0.5 to about 35 ppt (parts per thousand). Seawater is water from a sea, an ocean, or any other saline body of water that has a salinity ranging from about 35 to about 50 ppt. Brine can be water saturated or nearly saturated with salt. Brine could have a salinity that is about 50 ppt or greater. In some embodiments, the water source from which cations are derived is a mineral rich (e.g., calcium-rich and/or magnesium-rich) freshwater source. In some embodiments, the water source from which cations are derived is a naturally occurring saltwater source selected from a sea, an ocean, a lake, a swamp, an estuary, a lagoon, a surface brine, a deep brine, an alkaline lake, an inland sea, or the like. In some embodiments, the water source from which cations are derived is an anthropogenic brine selected from a geothermal plant wastewater or a desalination wastewater.

Freshwater is often a convenient source of cations (e.g., cations of alkaline earth metals such as Ca²⁺— and Mg²⁺). Any of a number of suitable freshwater sources may be used, including freshwater sources ranging from sources relatively free of minerals to sources relatively rich in minerals. Mineral-rich freshwater sources may be naturally occurring, including any of a number of hard water sources, lakes, or inland seas. Some mineral-rich freshwater sources such as alkaline lakes or inland seas (e.g., Lake Van in Turkey) also provide a source of pH-modifying agents. Mineral-rich freshwater sources may also be anthropogenic. For example, a mineral-poor (soft) water may be contacted with a source of cations such as alkaline earth metal cations (e.g., Ca²⁺, Mg²⁺, etc.) to produce a mineral-rich water that is suitable for methods and systems described herein. Cations or precursors thereof (e.g. salts, minerals) may be added to freshwater (or any other type of water described herein) using any convenient protocol (e.g., addition of solids, suspensions, or solutions). In some embodiments, divalent cations selected from Ca⁺² and Mg⁺² are added to freshwater. In some embodiments, monovalent cations selected from Na⁺ and K⁺ are added to freshwater. In some embodiments, freshwater is combined with combustion ash (e.g., fly ash, bottom ash, boiler slag), or products or processed forms thereof, yielding a solution comprising calcium and magnesium cations.

In some embodiments, an aqueous solution of cations may be obtained from an industrial plant that is also providing a combustion gas stream. For example, in water-cooled industrial plants, such as seawater-cooled industrial plants, water that has been used by an industrial plant for cooling may then be used as water for producing solutions, slurries, or solid precipitation material. If desired, the water may be cooled prior to entering a system of the invention. Such approaches may be employed, for example, with once-through cooling systems. For example, a city or agricultural water supply may be employed as a once-through cooling system for an industrial plant. Water from the industrial plant may then be employed for producing solutions, slurries, or precipitation material, wherein output water has a reduced hardness and greater purity.

In some embodiments, the aqueous solution contains carbonates and/or bicarbonates, which may be in combination with a divalent cation such as calcium and/or magnesium, or with a monovalent cation such as sodium.

In some embodiments, the aqueous solutions of the invention include a CO₂ sequestering additive. CO₂ sequestering additives are components that store a significant amount of CO₂ in a storage stable format (e.g. hydroxides or carbonates that upon reacting with CO₂ convert to carbonate or bicarbonate), such that CO₂ gas is not readily produced from the product and released into the atmosphere. In certain embodiments, the CO₂ sequestering additives can store 50 tons or more of CO₂, such as 100 tons or more of CO₂, including 250 tons or more of CO₂, for instance 500 tons or more of CO₂, such as 750 tons or more of CO₂, including 900 tons or more of CO₂ for every 1000 tons of composition of the invention. In certain embodiments, the CO₂ sequestering additives can store 20 tons or more for every 1000 tons of composition of the invention. In certain embodiments, the CO₂ sequestering additives can store 40 tons or more for every 1000 tons of composition of the invention. In certain embodiments, the CO₂ sequestering additives can store 45 tons or more for every 1000 tons of composition of the invention. For example, in some embodiments, the concentration of the CO₂ sequestering additive is about 1 to about 2 molar. In some applications these 1 molar solutions will be able to sequestered 1 ton of CO₂ in 22 tons of the aqueous solution. Thus if one will like to store 1000 tons of CO₂, 22,000 tons of aqueous solution will be needed. To use this amount of aqueous solution, according to the methods, apparatus and systems described herein, a container of about 30 meters on the side can be used. In certain embodiments, the CO₂ sequestering additives of the compositions of the invention comprise about 5% or more of CO₂, such as about 10% or more of CO₂, including about 25% or more of CO₂, for instance about 50% or more of CO₂, such as about 75% or more of CO₂, including about 90% or more of CO₂, e.g., present as one or more sequestering products (e.g. carbonate compounds).

In some embodiments the aqueous solution is an alkaline solution (e.g. NaOH). CO₂ may react with the alkaline solution to form a product (e.g., Na₂CO₃ or NaHCO₃).

Examples of aqueous solutions that can be used in the present invention include strongly alkaline hydroxide solutions like, for example, sodium and potassium hydroxide. Hydroxide solutions in excess of 0.1 molarity can readily remove CO₂ from air where it is bound, e.g., as a carbonate. Sodium hydroxide is a particular convenient choice. Organic amines are another example of aqueous solutions that may be used. Yet another choice of aqueous solutions includes weaker alkaline brines like sodium or potassium carbonate brines. The following discussion applies to all aqueous solutions that store CO₂ at least in part in an ionic carbonate or bicarbonate form.

### Methods and Systems

The invention provides for methods, systems, apparatus and compositions for extracting CO₂ from a gas stream. Examples of gas stream include, but are not limited to, ambient air and combustion of fuel.

In some embodiments, the methods for extracting CO₂ from a gas stream comprise bringing the gas stream in contact with a primary sorbent, releasing the carbon dioxide from the primary sorbent to create a CO₂-enriched gas mixture, and bringing the CO₂-enriched gas mixture in contact with an aqueous solution, wherein the aqueous solution absorbs CO₂ from the CO₂-enriched gas mixture.

FIG. 2 shows an embodiment of the invention. The primary sorbent is a humidity swing chamber. The sorbent rotates through ambient air collecting CO₂ as shown in step 1. The sorbent is then exposed to the humidity swing and it releases CO₂ into an air stream as shown in step 2. The humidity swing can be generated using, for example, water spray or water vapor or any other methods described herein. The air is enriched with CO₂ to 5% or more as shown in step 3. In some embodiments, the air is enriched to 10%, 15%, 20%, 30%, 40%, 50%, 60%, 70%, 80%, 90%, 95% or 100%. The CO₂ is carried in a closed air flow to an aqueous solution chamber containing the aqueous solution (e.g. brine) as shown in step 4. In some embodiments, the aqueous solution can be a brine pump from saline aquifer. In some embodiments, the brine may be fortified to ensure certain qualities, e.g., salinity and alkalinity. The CO₂ is absorbed into the aqueous solution as shown in step 5, while the CO₂ depleted air exits the aqueous solution chamber as shown in step 6. The depleted air can be recycled back into the humidity swing chamber. The aqueous solution with the sequestered CO₂ can then be flown to the end use, e.g. algae culture feeding or CO₂ storage in seawater. In some embodiments, after the CO₂ sequestering product has been depleted from the aqueous solution the remaining aqueous solution can be recycled.

The simplest implementation of the methods described herein is a container in which the primary sorbent is a humidity swing sorbent. The humidity swing sorbent releases CO₂ into a slow air stream. The CO₂ is carried to the sorbent material and in turn is absorbed into the aqueous solution (e.g. brine) by bubbling the gas mixture through it. FIG. 3 shows an exemplary embodiment in which enriched air is bubbled through the brine in a brine chamber.

In some embodiments, the carbon dioxide-enriched gas stream from the primary sorbent may be combined with aqueous solution in a number of different ways, including but not limited to, bubbling the enriched stream through the alkaline solution, using a semi-permeable membrane that separates the gas from the brine, or flowing the alkaline solution over a rough surface, such as a surface formed using a foam material like aquafoam. The wetted surfaces provide large areas on which CO₂ can be removed from the gas stream. FIG. 4 shows an exemplary embodiment in which CO₂ is transferred to brine through hydrophobic tubes in a brine chamber. FIG. 5 shows an exemplary embodiment in which CO₂ is transferred to brine through foam across which brine is dripped.

In some embodiments, surfaces over which the CO₂ is released are created and juxtaposed with surfaces over which the aqueous solution (e.g. brine) flows. The aqueous solution (e.g. brine) is flowed over materials close to the sorbent filters, but care is taken that the brine does not get in direct contact with the sorbent filter. In some embodiments, the contact is tightened by using foams, and foams with larger holes through them. The holes set a fixed pressure drop, which in turn allows for a steady, well defined flow through the foam structure. The CO₂ is released and moved through a foam block which is continuously being flushed with the aqueous solution (e.g. brine). It is also possible to carry into the foam (or any other structure) a mineral powder that is being dissolved in situ. The acidity is being maintained by flowing a CO₂ rich gas stream through the exchangers. By bringing the source and alkalinity in close proximity, we reduce pH swings and thus maintain a higher efficiency.

In some embodiments, the primary sorbent (e.g. resin) can be separated from the aqueous solution (e.g. brine) with a hydrophobic porous membrane, which makes it impossible for the aqueous solution (e.g. brine) to cross the membrane, but which allows the transfer of water vapor and CO₂ across the membrane. This is particularly useful, if the CO₂ is immediately transferred from a humidity swing resin into liquid water.

In some embodiments, the primary sorbent material could be constructed in a way that the inside of the material acts as the sorbent, whereas the outside is designed to be porous and highly hydrophobic. By layering the material in this fashion, it is possible to have the water get in close contact with the membrane. A triple layer is even more advantageous in some applications. The interior absorbs CO₂, and is subject to a humidity swing. It is separated from an outside hydrophilic layer by a thin porous hydrophobic layer. The outside layer is supposed to hold water (even if it is saline) but does not participate directly in the humidity swing.

However, the hydrophilic layer in effect allows one to collect water rapidly, which is then transferred to the inside via vapor transport. This in turn will cause the release of the CO₂ which could be collected directly in the outside layer. If the material is directly immersed into the aqueous solution (e.g. brine), then the outside layer is unnecessary, as no buffer is needed. Indeed in such a system there is an advantage in minimizing the amount of water that is absorbed, and a hydrophobic boundary limits the amount of water that is transferred. For such a system it is preferable to eliminate the outside hydrophilic layer.

The thickness of the hydrophobic layer has to be sufficient to prevent liquid from penetrating directly through the layer. Its thickness thus will be governed by the diameter of the pores in the hydrophobic layer. Different materials will have different pore sizes, the thickness must be a small multiple of the pore diameter. Thus, if pores are measured in microns, thicknesses would be measured in tens of microns. In some embodiments, the thickness will be 1, 2, 3, 4, 5, 10, 15, 20 times the pore diameter.

There are several ways of producing such bilayer or trilayer material. One is coating the material with different polymers or paints that create hydrophobic porous layers (e.g. spraying, painting, or vapor deposition). The other alternative is to produce the sorbent material first and then defunctionalize the outer layer, by removing its amine groups. Given a hydrophobic backbone in the polymer, this will result in a thin hydrophobic layer. Pores can be incorporated by for example including pore formers that can be removed by a strong base. Such treatments can be applied by exposing the resin to different chemicals for prescribed amount of times, long enough to penetrate the surface, short enough to avoid entering the core of the material. Another hydrophilic layer can be added by functionalizing the outer most layer once again.

FIGS. 6 to 8 show different views of a device that can be used with the methods, systems and compositions described herein. This exemplary device is capable of capturing and transferring to brine approximately 5 metric tons of CO₂ per day. Both chambers are approximately 4 meters×2.5 meters×2.5 meters in the pictured configuration. In these figures the brine chamber is configured as in the hydrophobic tubes example shown in FIG. 4.

In some embodiments, one approach to CO₂ sequestration with ocean water is to add alkalinity to the ocean water. The following technique combines carbon sequestration with carbon utilization involving algae. CO₂ is collected by the air capture device and transferred to the aqueous brine. The brine as it becomes more acidic dissolves minerals like serpentine that do not contain carbonates themselves. This controls the pH and raises the CO₂ content of the brine. In a subsequent step, the algae will extract the CO₂. The biomass production removes CO₂ and therefore results in an increase in pH of the brine, which in turn can force the precipitation of carbonate. This carbonate is collected and disposed of, while algae consume additional CO₂ to produce biomass. As a result approximately half of the CO₂ is used as fuel; the other half is removed and stored. The net outcome is a system that produces fuels and performs CCS in a combined system. The advantage of combining the two parts is that the combined system simplifies the transfer of CO₂ to algae or other organisms. The CO₂ consumption of the process is, however, increased, as the process not only delivers carbon for fuel, but also a comparable amount of CO₂ for sequestration. In most instances, a price for carbon is necessary to justify the additional CO₂ collection.

In some embodiments, the invention encompasses the disposal of the CO₂ in the aqueous solution (e.g. brine). In some embodiments, the use of divalent ions leads to solid precipitates. For example, the use of divalent ions in water leads to the precipitation of carbonate. The precipitates can be removed and can be disposed of. In another embodiment, carbonic acid is added to solid sources of carbonate which are acidified to bicarbonate. In most cases is the more soluble form and thus stays in solution. Thus, in some embodiments, the invention encompassed the addition of alkalinity in the form of Ca or Mg carbonate or similar ions which then could be discharged a) into the ocean, or b) into pore waters that allow for the geological storage of dissolved CO₂ underground.

In some embodiments, CO₂ is only temporarily transferred to an aqueous solution, but is then again removed. For example, CO₂ could be stored in sodium or potassium bicarbonate brines to create CO₂ enriched atmospheres. In another example, CO₂ acidified brines that yield there CO₂ (as bicarbonate) to algae or similar photosynthesizing aqueous organisms can be produced.

### Compositions

In some embodiments, the invention provides for compositions comprising aqueous solutions with a certain percentage of a selected gas-sequestering product (e.g. calcium carbonate). In some embodiments, the invention provides for compositions that include a selected gas sequestering product (e.g. CO₂ sequestering product), wherein the selected gas sequestering agent comprises a chemical element from a selected gas that was released from a gas mixture enriched for that selected gas (e.g. CO₂). In some embodiments, the invention provides compositions that include a selected gas sequestering product, wherein the selected gas sequestering product comprises a chemical element from a selected gas that was released from gas mixture enriched with certain relative element isotope composition. In some embodiments, the invention provides for compositions that include a selected gas sequestering product (e.g. CO₂ sequestering product), wherein the selected gas sequestering product comprises a chemical element from a selected gas that was released from gas mixture enriched for that selected gas (e.g. CO₂) and wherein the gas mixture is enriched with certain relative element isotope composition.

In some embodiments, the gas mixture is a low pressure gas mixture. Without intending to be limited to any theory, by reducing the pressure of the intermediate sweep gas it becomes possible to enrich the selected gas (e.g., CO₂) which tends accumulate to a particular partial pressure. In some implementations, where the selected gas is CO₂, the other gases play no active role and thus can be safely removed by partially evacuating the system. This will result in a much higher fraction of CO₂ in the gas stream. This fraction can approach 100%. In some embodiments it is important to maintain a controlled pressure gradient in the gas stream which can only be controlled by retaining a residual sweep gas. The sweep gas controls the pressure gradient and sweeps the CO2 where it will flow. In some embodiments this pressure gradient can be maintained by water vapor in which case a temperature gradient in the system will control this pressure. H₂O vapors are easily removed by condensation.

In some embodiments, the invention provides aqueous solutions with a certain percentage of a CO₂ sequestering product (e.g. calcium bicarbonate). By “CO₂ sequestering product” is meant that the product contains carbon derived from CO₂. For example, compositions according to aspects of the present invention contain carbon that was released in the form of CO₂ from a gas mixture released from a primary sorbent.

In certain embodiments, the carbon sequestered in a CO₂ sequestering composition is in the form of a carbonate compound. Therefore, in certain embodiments, compositions according to aspects of the subject invention contain carbonate compounds where at least part of the carbon in the carbonate compounds is derived from a gas mixture released from a primary sorbent. As such, production of compositions of the invention results in the placement of CO₂ into a storage stable form, e.g., a stable component of a composition comprising an aqueous solution. Production of the compositions of the invention thus results in the prevention of CO₂ gas from entering the atmosphere. The compositions of the invention provide for storage of CO₂ in a manner such that CO₂ is sequestered (i.e., fixed) in the composition does not become part of the atmosphere. Compositions of the invention keep their sequestered CO₂ fixed for substantially the useful life the composition, if not longer, without significant, if any, release of the CO₂ from the composition. As such, where the compositions are consumable compositions, the CO₂ fixed therein remains fixed for the life of the consumable, if not longer. In some embodiments, the compositions are designed as waste products that retain the sequestered CO₂ after they enter into a waste stream.

The CO₂ sequestering products of the invention may include one or more carbonate compounds. The amount of carbonate in the CO₂ sequestering product, as determined by coulometry using the protocol described in coulometric titration, may be 40% or higher, such as 70% or higher, including 80% or higher. In these embodiments, the carbonate content of the product may be as low as 10%. In some embodiments, the fraction of the CO₂ sequestering product in the aqueous solution could be about 1 to about 5% for dilute solutions, about 5% to about 20% for concentrated solutions.

The carbonate compounds of the CO₂ sequestering products may be metastable carbonate compounds that are precipitated from a water, such as a salt-water. The carbonate compound compositions of the invention include precipitated crystalline and/or amorphous carbonate compounds. Specific carbonate minerals of interest include but are not limited to: calcium carbonate minerals, magnesium carbonate minerals and calcium magnesium carbonate minerals. Calcium carbonate minerals of interest include, but are not limited to: calcite (CaCO₃), aragonite (CaCO₃), vaterite (CaCO₃), ikaite (CaCO₃.6H₂O), and amorphous calcium carbonate (CaCO₃.nH₂O). Magnesium carbonate minerals of interest include, but are not limited to: magnesite (MgCO₃), barringtonite (MgCO₃.2H₂O), nesquehonite (MgCO₃.3H₂O), lanfordite (MgCO₃.5H₂O) and amorphous magnesium calcium carbonate (MgCO₃.nH₂O). Calcium magnesium carbonate minerals of interest include, but are not limited to dolomite (CaMgCO₃), huntite (CaMg₃(CO₃)₄) and sergeevite (Ca₂Mg₁₁(CO₃)₁₃H₂O). In certain embodiments, non-carbonate compounds like brucite (Mg(OH)₂) may also form in combination with the minerals listed above. As indicated above, the compounds of the carbonate compound compositions are metastable carbonate compounds (and may include one or more metastable hydroxide compounds) that are more stable in saltwater than in freshwater, such that upon contact with fresh water of any pH they dissolve and reprecipitate into other fresh water stable compounds, e.g., minerals such as low-Mg calcite.

The CO₂ sequestering products of the invention are derived from, e.g., precipitated from, a water. As the CO₂ sequestering products are precipitated from a water, they may include one or more additives that are present in the water from which they are derived. For example, where the water is salt water, the CO₂ sequestering products may include one or more compounds found in the salt water source. These compounds may be used to identify the solid precipitations of the compositions that come from the salt water source, where these identifying components and the amounts thereof are collectively referred to herein as a saltwater source identifier. For example, if the saltwater source is sea water, identifying compounds that may be present in the precipitated solids of the compositions include, but are not limited to: chloride, sodium, sulfur, potassium, bromide, silicon, strontium and the like. Any such source-identifying or “marker” elements would generally be present in small amounts, e.g., in amounts of 20,000 ppm or less, such as amounts of 2000 ppm or less. In certain embodiments, the “marker” compound is strontium, which may be present in the precipitated incorporated into the aragonite lattice, and make up 10,000 ppm or less, ranging in certain embodiments from 3 to 10,000 ppm, such as from 5 to 5000 ppm, including 5 to 1000 ppm, e.g., 5 to 500 ppm, including 5 to 100 ppm. Another “marker” compound of interest is magnesium, which may be present in amounts of up to 20% mole substitution for calcium in carbonate compounds. The saltwater source identifier of the compositions may vary depending on the particular saltwater source employed to produce the saltwater-derived carbonate composition. Also of interest are isotopic markers that identify the water source. These markers are useful, for example, in the verification and accounting of the CO₂. This may be important in that alkalinity removed from seawater, actually may not have the desired carbon reduction. That is, the CO₂ that is attached to the alkalinity was already attached when the alkalinity was in the seawater, and thus the net effect was close to zero. Thus it would indeed be advantageous to have technologies that could monitor the CO₂ content of the well.

Depending on the particular aqueous solution, the amount of CO₂ sequestering product that is present may vary. In some instances, the amount of CO₂ sequestering product ranges from about 1% to about 5%, 5 to 75% w/w, such as 5 to 50% w/w including 5 to 25% w/w and including 5 to 10% w/w.

Compositions of the invention include compositions that contain carbonates and/or bicarbonates, which may be in combination with a divalent cation such as calcium and/or magnesium, or with a monovalent cation such as sodium. The carbonates and/or bicarbonates may contain carbon dioxide from a source of carbon dioxide; in some embodiments the carbon dioxide originates from a gas mixture released from a primary sorbent that has extracted CO₂ from ambient air, and thus some (e.g., at least 10, 50, 60, 70, 80, 90, 95%) or substantially all (e.g., at least 99, 99.5, or 99.9%) of the carbon in the carbonates and/or bicarbonates is of ambient origin. As is known, carbon of ambient air origin has a certain ratio of isotopes (¹³C and ¹²C) and thus the carbon in the carbonates and/or bicarbonates, in some embodiments, has a δ¹³C of, e.g., −10‰ to −7‰. Ambient air also has a certain fraction of the carbon in form of the ¹⁴C isotope, approximately 1.3 parts per trillion.

Compositions of the invention include a CO₂ sequestering additive as described above in the Aqueous Solution section.

In certain embodiments, compositions of the invention will contain carbon extracted from ambient air; because of its origin the carbon isotopic fractionation (δ¹³C) will have a certain value.

As is known in the art, the plants from which fossil fuels are derived preferentially utilize ¹²C over ¹³C, thus fractionating the carbon isotopes so that the value of their ratio differs from that in the atmosphere in general; this value, when compared to a standard value (PeeDee Belemnite, or PDB, standard), is termed the carbon isotopic fractionation (δ¹³C) value. δ¹³C values for coal are generally in the range −30 to −20‰ and δ¹³C values for methane may be as low as −20‰ to −40‰ or even −40‰ to −80‰. δ¹³C values for atmospheric CO₂ are −10‰ to −7‰, for limestone +3‰ to −3‰, and for marine bicarbonate, 0‰. Thus the δ¹³C values for the aqueous solution can be traced back to the CO₂ origin. Even when the aqueous solutions comprise other sources of carbon, e.g. natural limestone, the δ¹³C of the aqueous composition can be determined.

In some embodiments, the compositions of the invention includes a CO₂-sequestering product comprising carbonates, bicarbonates, or a combination thereof, in which the carbonates, bicarbonates, or a combination thereof have a carbon isotopic fractionation (δ¹³C) value less than 3‰. Compositions of the invention thus include an aqueous solution with a δ¹³C less than 2‰, less than 1‰, less than −5‰, less than −10‰, such as less than −12‰, −14‰, −16‰, −18‰, −20‰, −22‰, −24‰, −26‰, −28‰, or less than −30‰. In some embodiments the invention provides an aqueous solution with a δ¹³C less than −7‰. In some embodiments the invention provides an aqueous solution with a δ¹³C less than −10‰. In some embodiments the invention provides an aqueous solution with a δ¹³C less than −14‰. In some embodiments the invention provides an aqueous solution with a δ¹³C less than −18‰. In some embodiments the invention provides an aqueous solution with a δ¹³C less than −20‰. In some embodiments the invention provides an aqueous solution with a δ¹³C less than −24‰. In some embodiments the invention provides an aqueous solution with a δ¹³C less than −28‰. In some embodiments the invention provides an aqueous solution with a δ¹³C less than 3‰. In some embodiments the invention provides an aqueous solution with a δ¹³C less than 5‰. Such an aqueous solution may be carbonate-containing materials or products, as described above, e.g., an aqueous solution with that contains at least 10, 20, 30, 40, 50, 60, 70, 80, or 90% carbonate, e.g., at least 50% carbonate w/w.

The relative carbon isotope composition (δ¹³C) value with units of ‰ (per mille) can be measured of the ratio of the concentration of two stable isotopes of carbon, namely ¹²C and ¹³C, relative to a standard of fossilized belemnite (the PDB standard).

δ¹³C ‰=[(¹³C/¹²Cₛₐₘₚₗₑ−¹³C/¹²C_{PDB standard})/(¹³C/¹²C_{PDB standard})]×1000

In some embodiments the invention provides a method of characterizing a composition comprising measuring its relative carbon isotope composition (δ¹³C) value. In some embodiments the composition is a composition that contains carbonates, e.g., magnesium and/or calcium carbonates. Any suitable method may be used for measuring the δ¹³C value, such as mass spectrometry or off-axis integrated-cavity output spectroscopy (off-axis ICOS).

One difference between the carbon isotopes is in their mass. Any mass-discerning technique sensitive enough to measure the amounts of carbon we have can be used to find ratios of the ¹³C to ¹²C isotope concentrations. Mass spectrometry is commonly used to find δ¹³C values. Commercially available are bench-top off-axis integrated-cavity output spectroscopy (off-axis ICOS) instruments that are able to determine δ¹³C values as well. These values are obtained by the differences in the energies in the carbon-oxygen double bonds made by the ¹²C and ¹³C isotopes in carbon dioxide. The δ¹³C value of a carbonate precipitate from a carbon sequestration process serves as a fingerprint for a CO₂ gas source, as the value will vary from source to source, but in most carbon sequestration cases δ¹³C will generally be in a range of 3‰ to −35‰.

In some embodiments the methods further include the measurement of the amount of carbon in the composition. Any suitable technique for the measurement of carbon may be used, such as coulometry.

Precipitation material, which comprises one or more synthetic carbonates derived from ambient CO₂, reflects the relative carbon isotope composition (δ¹³C) of the ambient air. The relative carbon isotope composition (δ¹³C) value with units of ‰ (per mille) is a measure of the ratio of the concentration of two stable isotopes of carbon, namely ¹²C and ¹³C, relative to a standard of fossilized belemnite (the PDB standard).

δ¹³C ‰=[(¹³C/¹²Cₛₐₘₚₗₑ−¹³C/¹²C_{PDB standard})/(¹³C/¹²C_{PDB standard})]×1000

As such, the δ¹³C value of the CO₂ sequestering product serves as a fingerprint for a CO₂ gas source. The δ¹³C value may vary from source to source, but the δ¹³C value for composition of the invention generally, but not necessarily, ranges between 3‰ to −15‰. In some embodiments, the δ¹³C value for the CO₂ sequestering additive is between 1‰ and −50‰, between −5‰ and −40‰, between −5‰ and −35‰, between −7‰ and −40‰, between −7‰ and −35‰, between −9‰ and −40‰, or between −10‰ and −1‰. In some embodiments, the δ¹³C value for the CO₂ sequestering additive is less than (i.e., more negative than) 3‰, 2‰, 1‰, −1‰, −2‰, −3‰, −5‰, −6‰, −7‰, −8‰, −9‰, −10‰, −11‰, −12‰, −13‰, −14‰, −15‰, −16‰, −17‰, −18‰, −19‰, −20‰, −21‰, −22‰, −23‰, −24‰, −25‰, −26‰, −27‰, −28‰, −29‰, or −30‰, wherein the more negative the δ¹³C value, the more rich the synthetic carbonate-containing composition is in ¹²C. Any suitable method may be used for measuring the δ¹³C value, methods including, but no limited to, mass spectrometry or off-axis integrated-cavity output spectroscopy (off-axis ICOS).

In certain embodiments, compositions of the invention will contain carbon extracted from ambient air; because of its origin the carbon isotopic fractionation (¹⁴C) will have a certain value. The ¹⁴C fraction of the atmosphere is around 1.3 parts per trillion, i.e. 1.3 in a trillion carbon atoms are ¹⁴C atoms. The half-life of ¹⁴C is 5,730+40 years. It decays into nitrogen-14 through beta decay. As a result of this decay. coal, for example, has about 100 times less ¹⁴C than the atmosphere. Limestone is essentially free of ¹⁴C. As such, the ¹⁴C value of the CO₂ sequestering product or of the released CO₂ serves as a fingerprint for a CO₂ gas source. Even when the aqueous solutions comprise other sources of ¹⁴C, the ¹⁴C of the aqueous composition can be determined as the addition of ¹⁴C will be essentially atmospheric in level. Without intending to be limited to any theory, the fractionation during the process is of little importance, because the ¹⁴C content of the sources can vary greatly. In some cases the fingerprint can be complicated because the sorbent material or the sorbent brine can contain some carbonates that are of a different source and thus contain a different amount of ¹⁴C. However, after many cycles of use, the ¹⁴C released from the sorbent, or stored in the CO₂ sequestering product should be very close to the ¹⁴C content of the air, which is approximately 1.3 atoms for every one trillion ¹²C atoms.

In some embodiments, the compositions of the invention includes a CO₂-sequestering product comprising carbonates, bicarbonates, or a combination thereof, in which the carbonates, bicarbonates, or a combination thereof have a carbon isotopic fractionation of ¹⁴C of about 0.05 part per trillion to about 1 parts per trillion. Compositions of the invention, thus, include an aqueous solution with a carbon isotopic fractionation of ¹⁴C of about 0.05, 0.1, 0.5, 0.6, 0.7, 0.8, 0.9, 1, 1.1, 1.2, 1.3, 1.5 or 2 parts per trillion. Compositions of the invention, thus, include an aqueous solution with a carbon isotopic fractionation of ¹⁴C of about 1 parts per trillion. Compositions of the invention, thus, include an aqueous solution with a carbon isotopic fractionation of ¹⁴C of about 1.1 parts per trillion. Compositions of the invention, thus, include an aqueous solution with a carbon isotopic fractionation of ¹⁴C of about 1.3 parts per trillion.

In some embodiments, the compositions of the invention includes a CO₂-sequestering product comprising carbonates, bicarbonates, or a combination thereof, in which the carbonates, bicarbonates, or a combination thereof have a carbon isotopic fractionation of ¹⁴C of about 1 parts per trillion and a δ¹³C less than −7‰. In some embodiments, the compositions of the invention include a CO₂-sequestering product comprising carbonates, bicarbonates, or a combination thereof, in which the carbonates, bicarbonates, or a combination thereof have a carbon isotopic fractionation of ¹⁴C of about 1 parts per trillion and a δ¹³C less than −10‰. In some embodiments, the compositions of the invention include a CO₂-sequestering product comprising carbonates, bicarbonates, or a combination thereof, in which the carbonates, bicarbonates, or a combination thereof have a carbon isotopic fractionation of ¹⁴C of about 1 parts per trillion and a δ¹³C less than −14‰. In some embodiments, the compositions of the invention includes a CO₂-sequestering product comprising carbonates, bicarbonates, or a combination thereof, in which the carbonates, bicarbonates, or a combination thereof have a carbon isotopic fractionation of ¹⁴C of about 1 parts per trillion and a δ¹³C less than −18‰. In some embodiments, the compositions of the invention include a CO2-sequestering product comprising carbonates, bicarbonates, or a combination thereof, in which the carbonates, bicarbonates, or a combination thereof have a carbon isotopic fractionation of ¹⁴C of about 1 parts per trillion and a δ¹³C less than −20‰. In some embodiments, the compositions of the invention include a CO₂-sequestering product comprising carbonates, bicarbonates, or a combination thereof, in which the carbonates, bicarbonates, or a combination thereof have a carbon isotopic fractionation of ¹⁴C of about 1 parts per trillion and a δ¹³C less than −24‰. In some embodiments, the compositions of the invention include a CO₂-sequestering product comprising carbonates, bicarbonates, or a combination thereof, in which the carbonates, bicarbonates, or a combination thereof have a carbon isotopic fractionation of ¹⁴C of about 1 parts per trillion and a δ¹³C less than −28‰. In some embodiments, the compositions of the invention include a CO2-sequestering product comprising carbonates, bicarbonates, or a combination thereof, in which the carbonates, bicarbonates, or a combination thereof have a carbon isotopic fractionation of ¹⁴C of about 1 parts per trillion and a δ¹³C less than −3‰. In some embodiments, the compositions of the invention include a CO₂-sequestering product comprising carbonates, bicarbonates, or a combination thereof, in which the carbonates, bicarbonates, or a combination thereof have a carbon isotopic fractionation of ¹⁴C of about 1 parts per trillion and a δ¹³C less than −5‰. In any of these embodiments, the amount of CO₂ sequestering product ranges from about 1% to about 5%, 5 to 75% w/w, such as 5 to 50% w/w including 5 to 25% w/w and including 5 to 10% w/w.

In some embodiments, the compositions of the invention are used to store CO₂ in the ocean. In some embodiments, the compositions of the invention are used to feed algae cultures. In some embodiments, the compositions of the invention are used to store CO₂. In some embodiments, the compositions of the invention are used to dissolve alkali metals.

### Applications

a. Ocean Water

One implementation of the methods, apparatuses, systems and compositions described herein uses ocean water or mineral moistened by ocean water to produce a bicarbonate brine. CO₂ may be sequestered by converting carbonates to bicarbonate salts that are added to the ocean. This process may employ Na, K, Ca, or Mg, or any other suitable element as a cation. One can provide these cations in various forms. In some embodiments, the humidity swing process previously disclosed can be used to create either pure CO₂ at low pressure, or a mixture of air and CO₂, which is subsequently exposed to an aqueous solution produced by washing seawater over a suitable rock material to create a carbonate brine. Suitable rock materials could be, but are not limited to: serpentines, lime stone, magnesium carbonates, dolomites, or sodium and potassium rich clays or basalts. In each case we desire materials in which the weathering effect is substantial where a partial pressure of CO₂ of a fraction of an atmosphere is sufficient to lead to the absorption of most of the CO₂.

We propose to enrich seawater with as much bicarbonate as it is capable of leaching out of the mineral base and then promptly dilute it in the ocean. See FIG. 1. To this end we expose the seawater to a CO₂ enriched atmosphere. Adding 5% of an atmosphere of CO₂ will acidify the seawater sufficiently for it to dissolve limestone. Once the mineral has been dissolved, the pH will rise and thus create a bicarbonate brine that holds very little excess CO₂. Once this brine is mixed seawater, the bicarbonate will remain dissolved as the offstream is mixed with large volumes of seawater. To the extent the pH is still lower than in natural seawater, a slight excess of CO₂ will be released back to the atmosphere. The actual uptake rate of the seawater is determined by the amount of Ca that has been dissolved. Each mole of Ca dissolved will hold on to an additional amount of CO₂ that has the ratio of bicarbonate to carbonate as is normal in seawater with a pH around pH 8, and which carries a total charge equivalent of 2 moles. Hence the additional mole of Ca will sequester nearly two moles of CO₂. If the calcium source was a carbonate rock than for every mole of CO₂ added to the ocean half a mole of CO₂ would be derived from the limestone, and only the second half mole (actually closer to 0.45 moles) would be added from the air capture device.

In this design, seawater exposed to limestone, or other mineral rock becomes the secondary sorbent needed to complete the reaction. Referring to FIG. 1, seawater enriched in CO₂ may be poured over suitable rock materials that then dissolve. The exposure of the seawater to CO₂ either occurs before the seawater is used to extract calcium from a lime stone or during the dissolution process. Limestone does not dissolve into seawater at normal pH. For instance, lime slurry may be poured into a polyurethane foam structure, where CO₂ gas encounters lots of surface on its way through the device. The dissolving rock materials are also exposed to moisture, which is also used to humidify the sorbent resin. Thus, we are using the alkalinity-laden water as the secondary sorbent for our system. The carbonate-rich water which results is then returned to the ocean where it will be diluted, making any change in the ocean water chemistry barely perceptible.

It is also possible to directly use seawater to drive the dissolution, and the presence of carbonic anhydrase may speed up this process dramatically. See, e.g. PCT International Patent Appln. Serial No. PCT/US08/60672, assigned to a common assignee and incorporated by reference herein, for a discussion of the use of carbonic anhydrase to accelerate the CO₂ capture process.

The carbon dioxide-enriched gas stream from an air capture device may be combined with the alkaline sea water in a number of different ways, including but not limited to: bubbling the enriched stream through the alkaline solution, using a semi-permeable membrane that separates the gas from the liquid but permits the transfer of CO₂, or flowing the alkaline solution over a rough surface, such as a surface formed using various foam structures including aquafoam like structures that can contain large amounts of liquid.

An alternative method for capturing and sequestering CO₂ is to use clean water or a very dilute bicarbonate solution to free the CO₂ from the ion exchange resin, and bring this liquid in contact with a hydrophobic gas diffusion membrane with the alkaline solution on the opposite side of the membrane. The high partial pressure of CO₂ in the water, will drive the transfer of CO₂ across the separation membrane into the alkaline solution. This transfer again could be enhanced by the presence of carbonic anhydrase.

A particular form of membrane design, we propose is to reverse the standard membrane with hydrophilic pores and gas on both sides, into one with hydrophobic pores and aqueous solutions on both sides. Then again we propose to attach carbonic anhydrase to the pore openings so that one can accelerate the transfer of CO₂ from the liquid phase into the gas phase in the pore and back out into the liquid on the other side. Permeation rates for the membrane should be fast when compared to gas separation methods, as the diffusion of CO₂ inside the membrane should be a lot faster.

The dissolution of limestone with air captured CO₂ is analogous to a process in which the CO₂ comes from a power plant. The present disclosure provides a substantial advantage over using the CO₂ from a power plant in that we do not have to bring enormous amounts of lime stone to a power plant, or distribute the CO₂ from a power plant to many different processing sites, but that we can instead develop a facility where seawater, lime and CO₂ from the air come together more easily. One specific implementation would be to create a small basin that is periodically flushed with seawater. The CO₂ is provided by air capture devices located adjacent to or even above the water surface. Of particular interest are sites where limestone or other forms of calcium carbonate (such as empty mussel shells) are readily available as well. If we have calcium carbonate, seawater and air capture devices in one place, we can provide a way of disposing of CO₂ in ocean water without changing the pH of the water.

Indeed, it is possible to install such units adjacent a coral reef area by bringing additional limestone to the site or by extracting limestone debris near the reef. If the units operate in a slight ocean current upstream of the reef, they can generate conditions that are more suitable to the growth of the coral reef. Growth conditions can be improved by raising the ion concentration product of Ca⁺⁺ and CO₃⁻⁻. This product governs the rate of coral reef growth.

b. Dissolving Alkaline Metals

In some embodiments, the invention provide for methods, systems, apparatuses and compositions to dissolve various alkaline minerals. Examples of alkaline minerals include, but are not limited to, limestone, dolomite, serpentines, olivines, and peridotite rocks.

In some embodiments, atmospheric of CO₂ extracted from the primary sorbent (e.g. humidity swing) will acidify the aqueous solution (e.g. seawater) sufficiently for it to dissolve the alkaline mineral (e.g. limestone). Once the mineral has been dissolved, the pH will rise and thus create a bicarbonate brine that holds very little excess CO₂.

A major advantage of the invention described herein is that the primary sorbent and the aqueous solution can be tightly connected. There is no need for long pipelines shipping CO₂, i.e., the two systems can be tightly connected. In other words, the air capture releases acidity which is consumed by the minerals. In one embodiment, the invention encompasses air collectors feeding their CO₂ directly into a tailing pile. In one embodiment, the invention provides a practical option to utilize coastal limestone, to absorb carbonic acid.

c. Algae Cultures

In some embodiments of the invention, the CO₂ is extracted and delivered to an algal or bacterial bioreactor. This may be accomplished using conventional CO₂ extraction methods or by using an improved extraction method as disclosed herein; e.g., by a humidity swing. A humidity swing is advantageous for extraction of CO₂ for delivery to algae because the physical separation allows the use of any collector medium without concern about compatibility between the medium and the algae culture solution. The CO₂ extracted from the sorbent is then absorbed into an aqueous solution as described above. The aqueous solution is then feed to the algae. Nutrients can be added to the aqueous solution and it becomes the feed stock for algae. In some embodiment of the invention, the aqueous solution feed is not recycled, so that the aqueous solution becomes a consumable. In some embodiments, the aqueous solution is recycled. The aqueous solution is changed by the algae which will remove some CO₂ and some nutrients, and they will add some waste products. The process will also lose some water through evaporation. After removing the waste products, e.g., by filtration, and adding the missing nutrients and CO₂ and the aqueous solution could then be used again to feed algae. In some embodiments the aqueous solvent is a bicarbonate brine.

By feeding the bicarbonate brine to the algae, CO₂ can be removed from the brine without first converting the CO₂ back to CO₂ gas. Many algae can utilize bicarbonate as their carbon source. Also, some algae prefer bicarbonate over CO₂ as their carbon source. These are often algae that are indigenous to alkaline lakes, where inorganic carbon is predominantly present as bicarbonate. Some of these algae can tolerate large swings in pH of 8.5 up to 11. Other algae can utilize HCO₃⁻ as their carbon source, but require pH ranges below pH=9. In some embodiments, CO₂ would be bubbled through the bicarbonate/carbonate solution. In other embodiments, higher dilutions will have nearly entirely bicarbonate at a pH of about 8.

Algae use the carbon source to produce biomass through photosynthesis. Since photosynthesis requires CO₂ not bicarbonate, the algae catalyze the following reaction:

HCO₃⁻→CO₂+OH⁻

In the presence of HCO₃⁻, this becomes:

HCO₃⁻+OH→CO₃⁻²+H₂O

Algae growth in a bicarbonate solution induces the following changes in the solution: (1) a decrease in HCO₃⁻ concentration; (2) an increase in CO₃^{- 2}concentration; and (3) an increase in pH.

d. Storage

In certain embodiments, the invention provides for the storage of CO₂ As mention above, CO₂ is sequestered in the aqueous solutions. In some embodiments, the carbon sequestered in a CO₂ sequestering composition is in the form of a carbonate compound. Therefore, in certain embodiments, compositions according to aspects of the subject invention contain carbonate compounds where at least part of the carbon in the carbonate compounds is derived from a gas mixture released from a primary sorbent. As such, production of compositions of the invention results in the placement of CO₂ into a storage stable form, e.g., a stable component of a composition comprising an aqueous solution. Production of the compositions of the invention thus results in the prevention of CO₂ gas from entering the atmosphere. The compositions of the invention provide for storage of CO₂ in a manner such that CO₂ sequestered (i.e., fixed) in the composition does not become part of the atmosphere. Compositions of the invention keep their sequestered CO₂ fixed for substantially the useful life the composition, if not longer, without significant, if any, release of the CO₂ from the composition. As such, where the compositions are consumable compositions, the CO₂ fixed therein remains fixed for the life of the consumable, if not longer. In some embodiments, the compositions are designed as waste products that retain the sequestered CO₂ after they enter into a waste stream.

The CO₂ stored can be used for algae culture as described above, or for greenhouse applications as described in US publication number 2008/0087165. The compositions described herein can be used for temporary storage of CO₂ taken from the primary sorbent before it is processed further to concentrated, compressed or liquefied CO₂. It is worthwhile noting that a bicarbonate brine, for example, is a much cheaper way of storing intermediate product than holding CO₂ on a resin. In this case it is also possible to provide a clean brine that can be internally recycled, without getting in contact with large amounts of impurities.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1

### Device to Produce Alkaline Seawater

A device to produce a more alkaline seawater can be constructed by flowing seawater through a bed of serpentine. This seawater is then moved through foam structures through which CO₂ enriched air is flowing. The CO₂ enriched air is produced by using a device as shown in FIGS. 6 to 8: CO₂ sorbent filters, are exposed on a circular track to the wind. The individual filter boxes are collected into a shed like structure in which they are wetted and the CO₂ is released in response to the wetting of the resin. Air is slowly flowing through a small shed like structure in which resin filters are wetted and release their CO₂ back into the air stream. The Air speed in a particular implementation is about 0.6 m/s. The concentration of the CO₂ in gas stream can reach levels between 1 and 10%. In a preferred implementation it will be between 5 and 10%. The moist gas stream is then carried to another chamber of similar size, in which the gas stream could be simply bubbled through an alkaline brine, e.g. a sodium carbonate rich brine, that can contain a number of other ions, e.g. sodium chloride, as well as other impurities.

The uptake rate of carbonate/bicarbonate brines per unit of surface area and per unit of partial pressure are about 2 orders of magnitude slower than those of the resin surfaces. On the other hand, the partial pressure of CO₂ is about 2 orders of magnitude larger. Hence the uptake rates (in moles per square meter of surface) one can achieve per unit of area are quite comparable to those one can achieve in the regeneration of the air collector. Hence the transfer of the CO₂ into the brine is of a similar size than the release on the other side. Therefore as shown in the figures, the box for the gas to liquid transfer has about the same size as the resin release system. Air flow speeds are similar as well, and of course the total airflow is the same.

Another, more effective way of making contact between the liquid and the gas stream is to have the aqueous solution flow through a bed of Raschig rings that are sprayed with the aqueous solution on the top and which slowly flows through the packing until it emerges loaded with CO₂ on the bottom. Raschig rings could be sized at approximately 1 cm in diameter. A more compact version can be achieved by replacing the rings with foam blocks that are wetted on the top and liquid is withdrawn at the bottom of the chamber. Air flow may enter from the bottom through tubes that pass through the bottom tray in the chamber, that end above the liquid level at the bottom of the tray. Alternatively the air can be routed sideway through the foam blocks. Small holes are cut into the foam blocks to even out pressure drops between the two sides of the foam block. A small amount of channeling of air in this manner reduces overall challenging and assures an more even use of the foam.

The resulting brine can then be used for the utilities described herein.

### Example 2

### Device That Uses Resin Materials in Foam Form

The resin which is shaped in form of foam blocks with some channels letting some air bypass the foam is exposed to wind so that it absorbs CO₂. The foam blocks are then arranged within a chamber to release the CO₂ after wetting. The foam with larger holes passing through is exposed to a slow flowing air stream while water is flowing through its pores. The CO₂ stream is then carried by the air flow into a secondary foam structures through which an aqueous solution flows that is capable of taking up the CO₂. This process may repeat multiple times.

The brine to absorb the CO₂ can be adjusted in its concentration in several ways, depending on the goal of the process. For example, the solution can be adjusted so that bicarbonates are carried out of the foam and processed elsewhere, e.g. In an algal pond, or the solution is adjusted such that the input of CO₂ causes the precipitation of carbonates which are regularly washed out of the foam matrix. For example it is possible to start with a brine that is rich in Ca(OH)₂, which as it is carbonated in sufficiently high concentrations, will lead the precipitation of CO₂. The carbonate thus collected can be sequestered.

Another option is to use a highly concentrated sodium carbonate brine that after absorption of CO₂ will cause the precipitation of sodium bicarbonate. This in turn can be calcined, pure CO₂ is obtained and the sodium carbonate can be returned to the brine.

1-30. (canceled)

31. A method for extracting a selected gas from ambient air comprising bringing ambient air in contact with a primary sorbent; releasing the selected gas from the primary sorbent to create a selected gas-enriched gas mixture; and bringing the selected gas-enriched gas mixture in contact with an aqueous solution, by a process selected from the group consisting of:
(a) bubbling the selected gas-enriched gas mixture into the aqueous solution, wherein said aqueous solution is in a chamber into which aqueous solution enters at the top and from which a gas-enriched aqueous solution exits at the bottom;
(b) transferring the selected gas-enriched mixture through porous hydrophobic tubes; and
(c) exposing the selected gas-enriched gas mixture to a foam material comprising the aqueous solution;
wherein the aqueous solution absorbs gas from the selected gas-enriched gas mixture.

32. The method of claim 31, wherein the selected gas is selected from the group consisting of CO₂, NOₓ, and SO₂.

33. The method of claim 17, wherein the selected gas is CO₂.

34. The method of claim 31, wherein there is a gaseous gap between the primary sorbent and the aqueous solution.

35. The method of claim 31, wherein the aqueous solution does not come into direct contact with the primary sorbent material.

36. The method of claim 33, wherein the carbon dioxide-enriched gas mixture is brought in contact with the aqueous solution by bubbling the carbon dioxide-enriched gas mixture through the aqueous solution.

37. The method of claim 31, wherein the aqueous solution is flowed over surfaces that allow the aqueous solution to absorb carbon dioxide from the carbon dioxide-enriched gas mixture.

38. The method of claim 31, wherein the aqueous solution is water and is in contact with minerals from which alkali ions can be extracted.

39. The method of claim 38, wherein said water is undersaturated in carbonate ions.

40. The method of claim 38, wherein the water is continuously acidified with CO₂ in order to accelerate dissolution of alkali ions.

41. The method of claim 33, wherein the aqueous solution is an alkaline brine.

42. The method of claim 41, wherein said alkaline brine is formed by seawater that is held in contact with a rock material containing carbonate or other materials from which alkali ions can be leached during its exposure to the carbon dioxide.

43. The method of claim 42, wherein the leached ion is a calcium ion.

44. The method of claim 42, wherein at least part of the carbon dioxide is sequestered in the alkaline brine by forming carbonate ions, bicarbonate ions or a combination thereof, thereby neutralizing the aqueous solution, and further comprising returning the aqueous solution to its origin.

45. The method of claim 42, wherein the alkaline brine that sequesters carbon dioxide is discharged into a body of ocean water where it mixes with the ocean water and adds a stable bicarbonate salt that sequesters carbon dioxide.

46. The method of claim 33, wherein the primary sorbent is an ion exchange resin.

47. The method of claim 33, wherein the carbon dioxide-enriched gas mixture is brought in contact with the aqueous solution using a semi-permeable membrane that allows carbon dioxide to he transferred from the carbon dioxide-enriched gas mixture to the aqueous solution.

48. The method of claim 33, wherein the carbon dioxide is transferred into a first aqueous wash which is separated from the aqueous solution by a gas diffusion membrane which allows the transfer of carbon dioxide from one side of the gas diffusion membrane to the other.

49. The method of claim 31, wherein the aqueous solution is contained in or flows through a sponge or foam.

50. A composition comprising a CO₂ sequestering product, wherein the CO₂ sequestering product comprises carbon from ambient CO₂ from a gas mixture released from a primary sorbent.

51. The composition according to claim 50, wherein the CO₂ sequestering product is a carbonate compound composition, a hydroxide composition, a bicarbonate composition, or a mixture thereof.

52. The composition according to claim 51, wherein the carbonate compound composition comprises a precipitate from an alkaline-earth metal-containing water.

53. The composition of claim 51, wherein the δ¹³C is about 3% to about −35‰.

54. The composition of claim 51, wherein the ¹⁴C isotopic fraction is about 0.05 parts per trillion to about 2 parts per trillion.

55. The composition of claim 51, wherein the CO₂ sequestering product ranges from about 1% to about 5% w/w.

56. The composition of claim 51, wherein the CO₂ sequestering product ranges from about 5 to 75% w/w.

57. The composition of claim 50, wherein the percentage of CO₂ in said gas mixture is about 1% to about 10%.

58. The composition of claim 50, wherein the percentage of CO₂ in said gas mixture is about 90% to about 100%.

59. The composition of claim 50, wherein the composition is used to store CO₂, feed algae, or dissolve alkaline metals.

60. The composition of claim 50, wherein the composition is used to store CO₂ in the ocean.

61. A new composition comprising a consumable carbon sequestering product, said consumable carbon sequestering product comprising a metastable carbonate compound that is more stable in saltwater than in freshwater, wherein one or more ratios of carbon isotopes in said metastable carbonate compound reflects the relative isotope composition of ambient air.

62. The composition of claim 61, wherein the metastable carbonate compound has a δ¹³C of −10‰ to −3‰.

63. The composition of claim 61, wherein the metastable carbonate compound has a δ¹³C of about 0.05 parts per trillion to about 1 part per trillion.

64. The composition of claim 61, wherein the metastable carbonate compound is a calcium carbonate, and a magnesium carbonate, or a calcium magnesium carbonate.

65. The composition of claim 61, wherein the metastable carbonate compound is selected from the group consisting of: aragonite, vaterite, ikaite, amorphous calcium carbonate, barringtonite, nesquehonite, lansfordite, huntite, and sergeevite.

66. A method for producing a metastable carbonate compound, comprising capturing CO₂ from ambient air using a sorbent; releasing CO₂ from said sorbent; and precipitating a metastable carbonate compound from a solution comprising CO₂ released from said sorbent, wherein said metastable carbonate compound is more stable in saltwater than in freshwater.

67. The method of claim 66, wherein one or more ratios of carbon isotopes in said metastable carbonate compound reflects the relative isotope composition of ambient air.

68. The method of claim 67, wherein the metastable carbonate compound has a δ¹³C of −10‰ to −3‰.

69. The method of claim 67, wherein the metastable carbonate compound has a carbon isotopic fraction of ¹⁴C of about 0.05 parts per trillion to about 1 part per trillion.

70. The method of claim 66, wherein the metastable carbonate compound is a calcium carbonate, a magnesium carbonate, or a calcium magnesium carbonate.

71. The method of claim 66, wherein the metastable carbonate compound is selected from the group consisting of: aragonite, vaterite, ikaite, amorphous calcium carbonate, barringtonite, nesquehonite, lansfordite, huntite, and sergeevite.

72. The method of claim 66, wherein the sorbent is an anion exchange material.

73. The method of claim 66, wherein CO₂ is released from said sorbent by wetting said sorbent with liquid water or water vapor.

74. A method for the capture of CO₂ from air comprising the steps of: exposing a coated sorbent to ambient air, wherein said coated sorbent comprises a sorbent and a coating comprising a membrane that is hydrophobic and gas-permeable; and releasing CO₂ captured by said coated sorbent by immersing said coated sorbent in an algae culture comprising an alkaline brine.
